(19) United States
(12) Patent Application Publication
(10) Pub. No.: US 2015/0164608 A1
(43) Pub. Date: Jun. 18, 2015

(54) ACCURACY OF NAVIGATING A MEDICAL DEVICE

(71) Applicant: Markus Bartenstein, Munich (DE)

(72) Inventor: Markus Bartenstein, Munich (DE)

(73) Assignee: Brainlab AG, Feldkirchen (DE)

(21) Appl. No.: 14/405,412

(22) PCT Filed: Jun. 5, 2012
(86) PCT No.: PCT/EP2012/060540

§ 371 Date: Dec. 4, 2014

(51) Int. Cl. A61B 19/00

(52) U.S. Cl. CPC A61B 19/5244; A61B 19/20; A61B 19/54; A61B 2019/5257; A61B 2019/5274; A61B 2019/5265; A61B 2019/5287; A61B 2019/5437

(57) ABSTRACT

A data processing method of determining the position, in particular location and/or orientation, of a medical device relative to a multitactic detection device for detecting the position of the medical device, the data processing method being executed by a computer and comprising the following steps:
a) acquiring medical device position data comprising medical device position information describing a position of the medical device relative to a detection surface in which the detection device is located;
b) acquiring detection device geometry data comprising detection device geometry information describing a geometry of the detection device;
c) acquiring detection device geometry uncertainty data comprising detection device geometry uncertainty information describing a detection device geometry uncertainty associated with the geometry of the detection device described by the detection device geometry information, wherein the detection device geometry uncertainty is defined relative to an uncertainty surface;
d) acquiring surface relative position data comprising surface relative position information describing the relative position between the detection surface and the uncertainty surface;
e) determining, based on the medical device position data and the detection device geometry data and the detection device geometry uncertainty data and the surface relative position data, medical device position uncertainty data comprising medical device position uncertainty information describing a medical device position uncertainty associated with the position of the medical device, wherein the medical device position uncertainty is defined relative to the uncertainty surface.

The present invention is directed to a data processing method of determining the position, in particular the location and/or orientation of a medical device in accordance with claim 1, a corresponding computer program and navigation system configured to execute the data processing method.

In navigated surgical procedures, in particular in image guided surgery, it is often envisaged to determine the position of body parts or medical devices based on electromagnetic detection of markers. However, it is conventionally difficult to determine the error associated with determining such a position since a number of factors influence the accuracy with which these positions may be determined. In general, it is difficult to quantify these factors. If the errors associated with position determination remain unknown, this may lead to undesirable effects, in particular carrying out a surgical procedure based on such erroneous information may result in harming the patient or impairing the desired medical outcome.

The present invention therefore seeks to improve the accuracy with which the error of position determination for a navigated surgical procedure may be determined.

This problem is solved by the subject-matter of any appended independent claim. Advantages, advantageous features, advantageous embodiments and advantageous aspects of the present invention are disclosed in the following and contained in the subject-matter of the dependent claims. Different advantageous features can be combined in accordance with the invention as long as technically sensible and feasible. In particular, a feature of one embodiment which has the same or similar function of another feature of another embodiment can be exchanged. In particular, a feature of one embodiment which supplements a further function to another embodiment can be added to the other embodiment.

Preferably, the inventive method is at least partly executed by a computer. That is, all steps or just some of the steps (i.e. less than a total number of steps) of the inventive method may be executed by a computer.

It is the function of a marker to be detected by a marker detection device (for example, a camera or an ultrasound receiver), such that its spatial position (i.e. its spatial location and/or alignment) can be ascertained. The detection device is in particular part of a navigation system. The markers can be active markers. An active marker can for example emit electromagnetic radiation and/or waves, wherein said radiation can be in the infrared, visible and/or ultraviolet spectral range. The marker can also however be passive, i.e. can for example reflect electromagnetic radiation in the infrared, visible and/or ultraviolet spectral range. To this end, the marker can be provided with a surface which has corresponding reflective properties. It is also possible for a marker to reflect and/or emit electromagnetic radiation and/or waves in the radio frequency range or at ultrasound wavelengths. A marker preferably has a spherical and/or spheroid shape and can therefore be referred to as a marker sphere; markers can also, however, exhibit a cornered—for example, cubic—shape.

The method in accordance with the invention is in particular a data processing method. The data processing method is preferably performed using technical means, in particular a computer. In particular, the data processing method is executed by or on the computer. The computer in particular comprises a processor and a memory in order to process the data, in particular electronically and/or optically. The calculating steps described are in particular performed by a computer. Determining or calculating steps are in particular steps of determining data within the framework of the technical data processing method, in particular within the framework of a program. A computer is in particular any kind of data processing device, in particular electronic data processing device. A computer can be a device which is generally thought of as such, for example desktop PCs, notebooks, netbooks, etc., but can also be any programmable apparatus, such as for example a mobile phone or an embedded processor. A computer can in particular comprise a system (network) of “sub-computers”, wherein each sub-computer represents a computer in its own right. The term of computer encompasses a cloud computer, in particular a cloud server. The term of cloud computer encompasses cloud computer system in particular comprises a system of at least one cloud computer, in particular plural operatively interconnected cloud computers such as a server farm. Preferably, the cloud computer is connected to a wide area network such as the world wide web (WWW). Such a cloud computer is located in a so-called cloud of computers which are all connected to the world wide web. Such an infrastructure is used for cloud computing which describes computation, software, data access and storage services that do not require end-user knowledge of physical location and configuration of the computer that delivers a specific service. In particular, the term “cloud” is used as a metaphor for the internet (world wide web). In particular, the cloud provides computing infrastructure as a service (IaaS). The cloud computer may function as a virtual host for an operating system and/or data processing application which is used for executing the inventive method. Preferably, the cloud computer is an elastic compute cloud (EC2) provided by Amazon Web Services™. A computer in particular comprises interfaces in order to receive or output data and/or perform an analogue-to-digital conversion. The data are in particular data which represent physical properties and/or are generated from technical signals. The technical signals are in particular generated by means of (technical) detection devices (such as for example devices for detecting marker devices) and/or (technical) analytical devices (such as for example devices for performing imaging methods), wherein the technical signals are in particular electrical or optical signals. The technical signals represent in particular the data received or outputted by the computer.

The expression “acquiring data” encompasses in particular (within the framework of a data processing method) the scenario in which the data are determined by the data processing method or program. Determining data in particular encompasses measuring physical quantities and transforming the measured values into in particular digital data and/or computing the data by means of a computer, in particular computing the data within the method of the invention. The meaning of “acquiring data” in particular also encompasses the scenario in which the data are received or retrieved by the data processing method or program, for example from another program, a previous method step or a data storage medium, in particular for further processing by the data processing method or program. Thus, “acquiring data” can also for example mean waiting to receive data and/or receiving the data. The received data can for example be inputted via an interface. “Acquiring data” can also mean that the data processing method or program performs steps in order to (actively) receive or retrieve the data from a data source, for instance a data storage medium (such as for example a ROM, RAM, database, hard disc, etc.) or via the interface (for instance, from another computer or a network). The data can achieve the state of being “ready for use” by performing an additional step before the acquiring step. In accordance with this additional step, the data are generated in order to be acquired. The data are in particular detected or captured (for example, by an analytical device). Alternatively or additionally, the data are inputted in accordance with the additional step, for instance via interfaces. The data generated can in particular be inputted (for instance, into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention. Thus, “acquiring data” can also involve commanding a device to obtain and/or provide the data to be acquired. The acquiring step in particular does not involve an invasive step which would represent a substantial physical interference with the body requiring professional medical expertise to be carried out and entailing a substantial health risk even when carried out with the required professional care and expertise. Acquiring, in particular determining, data in particular does not involve a surgical step and in particular does not involve a step of treating a human or animal body using surgery or therapy. This also applies in particular to any steps directed to determining data. In order to distinguish the different data used by the present method, the data are denoted (i.e. referred to) as “XY data” and the like and are defined by the information which they describe which is preferably called “XY information”.

The invention also relates to a program which, when running on a computer or when loaded onto a computer, causes the computer to perform one or more or all of the method steps described herein and/or to a program storage medium on which the program is stored (in particular in a non-transitory form) and/or to a computer on which the program is running or into the memory of which the program is loaded and/or to a signal wave, in particular a digital signal wave, carrying information which represents the program, in particular the aforementioned program, which in particular comprises code means which are adapted to perform any or all of the method steps described herein.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, in particular computer-readable data storage medium comprising computer-usable, in particular computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, in particular a data processing device comprising a digital processor (central processing unit—CPU) which executes the computer program elements and optionally a volatile memory (in particular, a random access memory—RAM) for storing data used for and/or produced by executing the computer program elements. Within the framework of the present invention, a computer-usable, in particular computer-readable data storage medium can be any data storage medium which can include, store, communicate, propagate or transport the program for use on or in connection with the instruction-executing system, apparatus or device. The computer-usable, in particular computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. Preferably, the data storage medium is a non-volatile data storage medium. The computer program product and any software and/or hardware described here form the various means for performing the functions of the invention in the example embodiments. The computer and/or data processing device can in particular include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or vibration element incorporated into an instrument).

A navigation system, in particular a surgical navigation system, is understood to mean a system which can comprise: at least one marker device; a transmitter which emits electromagnetic waves and/or radiation and/or ultrasound waves; a receiver which receives electromagnetic waves and/or radiation and/or ultrasound waves and which embodies the aforementioned detection device; and an electronic data processing device which is connected to the receiver and/or the transmitter, wherein the data processing device (for example, a computer) in particular comprises a processor (CPU), a working memory, advantageously an indicating device for issuing an indication signal (for example, a visual indicating device such as a monitor and/or an audio indicating device such as a loudspeaker and/or a tactile indicating device such as a vibrator) and advantageously a permanent data memory, wherein the data processing device processes navigation data forwarded to it by the receiver and can advantageously output guidance information to a user via the indicating device. The navigation data can be stored in the permanent data memory and for example compared with data stored in said memory beforehand. The computer is connected to the detection device by a data interface for receiving the medical device position data from the detection device and for supplying that data to the computer. The navigation system further preferably includes a user interface for receiving data from the computer in order to provide information to a user, wherein the received data are generated by the computer on the basis of the results of the processing performed by the computer.

The inventive method preferably is a data processing method and serves determining the position of a medical device relative to a multi-tactic detection device. The term of position encompasses at least one of the location and the orientation of the medical device. The location and orientation are preferably described in a reference system, in particular a three-dimensional and preferably rectangular or spherical coordinate system, wherein the orientation is preferably additionally characterized by at least one in particular three-dimensional angle (i.e. three angles which are oriented along the primary directions of a coordinate system) between a characteristic dimension of the medical device and a characteristic dimension of the multi-tactic detection device. A multi-tactic detection device in particular is a detection device operating on the principle of detecting electromagnetic waves emitted or reflected by or from markers, wherein the radiation is received by the detection device at at least two discrete locations. An example of a multi-tactic detection device is a stereotactic detection device which has two detection units comprising in particular detection sensors and optical apertures at detection locations for receiving for example infrared radiation. Such a device is for example embodied by a stereotactic infrared camera. Within the framework of this disclosure, it is envisaged to also use detection devices having more than two locations of detection, for example four locations which are arranged on the corners of a rectangular two-dimensional shape such as a rectangle, in particular a square. The detection device is configured to detect a position of the medical device by receiving and evaluating the physical properties of the electromagnetic radiation emitted or reflected by or from the medical device or markers attached to the medical device, respectively. The medical device can be any device used or usable during a medical procedure, in particular a surgical procedure. The medical device therefore can be embodied by a medical instrument such as a scalpel, a syringe or a catheter. The medical device may also be embodied by tools such as a pointing device (pointer), constituents of a navigation system such as a computer, or a marker device (in particular, a reference star).

Within the inventive method, medical device position data is preferably acquired which comprises medical device position information. The medical device position information in particular describes a position of the medical device relative to a detection surface in which the detection device is located. The detection surface in particular is a surface, more particularly a plane, in which the detection locations of the detection device are located. If the detection device is embodied by a stereotactic camera, the detection surface in particular is the plane in which the optical apertures of the lenses are located or in which other parts of the detection units (in particular, the detection sensors such as CMOS-sensors or CCD-sensors) are located. Preferably, the detection surface is perpendicular, i.e. is positioned at a right angle, relative to a viewing direction of the detection device in which the detection device receives signals to be detected. If the distance between the detection locations is known, it is possible to determine the position of the medical device relative to the detection surface on the basis of fundamental geometric principles. In particular, if one assumes that the common plane in which the medical device and the detection device are located is located with a right angle relative to the detection surface, an electromagnetic signal received at each of the detection locations is preferably used to determine an azimuth of the position of the medical device relative to for example a centre line which preferably runs through the centre between the detection locations and preferably is parallel or lies in the object surface (in particular, in an object plane). The object surface is understood to be the surface, in particular plane, in which the positions of both the medical device and the detection device are located. Therefore, both a direction and a distance of the position of the medical device relative to the position of the detection device can be determined.

Preferably, detection device geometry data is acquired which comprises detection device geometry information. The detection device geometry information in particular describes a geometry of the detection device, more particularly a geometry of those parts of the detection device which lie in the detection surface. In particular, the detection device geometry information describes a distance between the detection locations. Additionally, the detection device geometry information may describe in particular the dimensions of the detection locations themselves such as the optical aperture, in particular the diameter of the object lenses or a shutter located in front of the detection units or the dimensions of a digital chip used as a detection sensor in a detection unit.

Preferably, detection device geometry uncertainty data is acquired which comprises detection device geometry uncertainty information. The detection device geometry uncertainty information in particular describes a detection device geometry uncertainty which is an uncertainty associated with the geometry of the detection device as it is described by the detection device geometry information. Preferably, the medical device geometry uncertainty is defined relative to an uncertainty surface. The detection device geometry uncertainty for example is an uncertainty associated with at least one of the acquired value of the distance between the detection locations and an uncertainty associated with the value of the object lens dimensions or the optical aperture. The detection device geometry uncertainty can be expressed in absolute values, for example as a maximum and/or minimum deviation of the given dimensions, in particular length dimensions, from their nominal value, in particular from their design value. Alternatively or additionally, the detection device geometry uncertainty may be expressed as a relative deviation of the mentioned quantities from their nominal value. The uncertainty surface preferably coincides with the object surface or is at least parallel to the object surface. In particular, the uncertainty surface is an uncertainty plane and, just like the object surface, preferably is oriented to be perpendicular relative to the detection surface.

Preferably, surface relative position data is acquired which comprises surface relative position information. The surface relative position information in particular describes the relative position (in particular, the orientation) between the detection surface and the uncertainty surface. For example, the relative position between the two surfaces (in particular, the orientation of the two surfaces towards each other) is described by an angle which preferably is a right angle between the two surfaces. According to a specific embodiment of the invention, the surface relative position information additionally also describes a relative position between the object surface and the uncertainty surface, in particular it describes an angle enclosed by the two surfaces. Therefore, the surface relative position information can give a complete characterization of the relative position of the surfaces in which the detection locations on the one hand and the detection locations and the medical device on the other hand are located.

Preferably, medical device position uncertainty data is determined based on the medical device position data and the detection device geometry data and the detection device geometry uncertainty data as well as based on the surface relative position data. The medical device position uncertainty data comprises medical device position uncertainty information which in particular describes a medical device position uncertainty. The medical device position uncertainty in particular describes an uncertainty associated with a position of the medical device and more particularly indicates the measure of uncertainty for the position of the medical device in absolute terms or as a relative quantity. In particular, the medical device position uncertainty may be expressed as a maximum and/or minimum absolute or relative deviation of the position of the medical device from its nominal value, in particular its determined, more particularly measured value. The nominal value in particular is the value which is determined based on the detection of electromagnetic radiation by the detection device. The medical device position uncertainty preferably is defined relative to the uncertainty surface, in particular it is defined to lie in the uncertainty surface. The uncertainty surface in which the medical device position uncertainty is defined preferably is the same uncertainty surface as the one in which the detection device geometry uncertainty is defined.

Preferably, the orientation of the medical device relative to the detection device, in particular relative to the detection surface, is determined based on acquiring the medical device position data for at least two detection features of the medical device. A detection feature of the medical device is understood to be an individual, discrete point and/or predetermined two-dimensional geometric feature in particular on the surface of the medical device, i.e. a surface feature of the medical device, which is suitable for detecting and preferably identifying the medical device by using a navigation system operating in particular on the principle of optical navigation. Examples of detection features include retroreflective markers (e. g. spherical markers), patterns of retroreflective foil and characteristic geometric features (e. g. for identification of the device by segmentation of image data taken e. g. by a video camera). Preferably, the position of each detection feature is determined individually within the framework of the inventive method. The detection feature may for example comprise markers having a predetermined spatial relationship to the detection features. In particular, the detection feature may be the position of a marker and therefore be part of the marker. Specific unique spatial arrangements of such markers may also be used to identify a type of the medical device or even an individual medical device among a plurality of medical devices belonging to the same type. If at least the type of the medical device has been identified, the inventive method may in particular acquire medical device geometry data comprising medical device geometry information which describes the geometry and in particular characteristic dimensions, more particularly the location of a longitudinal axis of the device, relative to the arrangement of markers. Based on the medical device geometry data and the medical device position data, the orientation of the medical device relative to the detection device may then be determined.

Preferably, determining the medical device position uncertainty data comprises determining the uncertainty, in at least one dimension and preferably two dimensions, of the position of the at least two detection features of the medical device. The medical device position uncertainty therefore in particular is determined based on individually determining the uncertainty of the position of each of the detection features. Since the position is preferably determined by three-dimensional coordinates while the location of the uncertainty surface and/or the object surface relative to the detection surface is known, the uncertainty is preferably determined in one or two dimensions (representing the free parameters of the positions of the detection features), may, however, also be determined in all three dimensions.

Preferably, an uncertainty of the orientation is determined based on the medical device position uncertainty data. The orientation is in particular defined as an angle, in particular an azimuthal angle, of the medical device relative to the detection surface and/or a center line between the detection locations, wherein the centre line preferably runs perpendicular to the detection surface in the centre (middle) between the detection locations. Once the medical device position uncertainty data is known for each individual detection feature, the uncertainty associated with the geometric quantities characterizing the orientation can be determined by known approaches such as in particular propagation of uncertainty according to Gauss.

The orientation is in particular defined by at least one angle between a straight line connecting the detection features and at least one of a direction parallel to the detection plane and a direction perpendicular to the detection plane. The uncertainty of the orientation is therefore determined for the at least one angle between the straight line and at least one of those two directions.

Preferably, the position of the medical device relative to the detection device is changed based on the medical device position uncertainty data, in particular in order to minimize the medical device position uncertainty. In particular, the inventive method may acquire information about an acceptable predetermined value of medical position uncertainty to which the determined value of the medical device position uncertainty preferably is compared. In particular if the determined uncertainty is larger than the acceptable uncertainty, the position of the medical device relative to the detection device is changed such that, for further measurement of the medical device position data, the medical device position uncertainty is decreased, preferably minimized. This feature supports an operator in using the medical device such that, during the envisaged medical procedure, position determination of the medical device is optimized. To this end, a navigation system used for implementing the inventive method preferably outputs guidance data comprising guidance information which describes visual and/or acoustic information output to the operator which tells him how to change the position of the medical device relative to the detection device in order to decrease the medical device position uncertainty.

Preferably, a graphical representation of the medical device position uncertainty information is output in particular by a monitor connected with a navigation system used for implementing the inventive method. The graphical representation may be embodied by an in particular multi-colour display showing for example a color bar or (absolute or relative) values describing the medical device position uncertainty which may be in addition highlighted by use of specific colours, such as for example red for a determined medical device position uncertainty which is higher than the acceptable medical device uncertainty, and green for a determined medical device position uncertainty which is lower than or equal to the acceptable device position uncertainty.

Preferably, the medical device position uncertainty data is determined based on acquiring the medical device position data for at least two positions of the medical device relative to the detection device, wherein the medical device position uncertainty data determined for one of those positions is compared to the medical device position uncertainty data determined for another one of those positions. Based on the outcome of the comparison, the position for which the medical device position uncertainty information indicates that the medical device position uncertainty is lower may be chosen as the position of the medical device relative to the detection device at which the medical device shall be used as envisaged for the specific medical procedure. This feature supports proper positioning of the detection device relative to the medical device such that the medical device position uncertainty is decreased, in particular minimized, for the envisaged medical procedure. To this end, the medical device position uncertainty data determined for the different positions of the medical device relative to the detection device may be sorted and preferably also displayed to an operator in an incremental, in particular an increasing or decreasing, order. In this way, an optimal position of the medical device relative to the detection device may be determined which in particular is a position for which a minimum of the medical device position uncertainty has been determined.

Preferably, weights (weighting factors) are applied to the medical device position uncertainty depending on the position of the medical device uncertainty information in the order. Besides assisting in finding an optimal position for conducting the envisaged procedure, determining the medical device position uncertainty for different operators, in particular surgeons, and sorting them in the aforementioned order supports creation of a ranking displaying the skill of the individual operator in application of a navigated medical (in particular, surgical) procedure. The ranking may be established for a single operator for example to keep track of his own performance (individual ranking) or for a plurality of operators for example to compare their performance (community ranking). The skill is then in particular defined as the ability to find an optimal positioning of the detection device relative to an intraoperative situs, in particular relative to the medical device (more particularly, when it placed at its desired location of use) and thus describes the operator's ability in creating a suitable operating environment.

In general, the present disclosure is directed to a method of acquiring values for specific input parameters associated with a navigated medical procedure, computing certain characteristics of such input data and outputting an improvement suggestion and preferably a ranking.

As input parameters, the following quantities are envisaged:
orientation of the camera with respect to a reference star
distance camera to a reference star
distance from a reference star to the patient head
threshold values used during registration
location of points acquired during a specific task (e.g. during registration)
number of verifications of the registration (e.g. by holding a pointer still)
reliability map used to control the data
type of image data set used (e.g. CT or MR)
quality of the dataset used for the surgery (e.g. are all relevant body parts available, resolution sufficient)
number of registrations that had to be performed in order to get an acceptable result
number of interactions the customer had with the software (e.g. total number of touch events)
was a specific feature used or not (e.g. was the laser button pressed during set up?)
frequency/duration a specific feature was used (e.g. how long was the motorized joint pressed to align the camera?)
picture of the OR setup (e.g. taken via the tracking cameras)
length of time needed for setting up the system
length of time the system was in idle mode, i.e. no interaction with the customer took place
where are the devices and key components located with respect to each other and or with respect to other components in the OR (would require techniques to locate the system and component position within the OR)
which tool geometries have been used
type of used markers
data and time when the last service was performed (entered by support)

Most of the above-mentioned parameters could be permanently logged or logged only during a specific tasks or time interval (e.g. during registration). Note that some of the above-mentioned information is already part of the statistical log file used by Brainlab® software applications.

The computation relies on the application of specific assessment criteria that weight the different input parameters. The weighting factors on the other hand depend on the individual application, in particular the medical procedure which is to be carried out. In the following, two examples are given for the case of different surgical applications in the field of neurosurgery and orthopaedics.

In neurosurgery each surgery is typically a unique and individual case. During these surgeries precision is the key. Thus the actual accuracy of the system at a given time is one but not the only important number. To determine the accuracy with respect to the ideal case the following weighting factors could be used with decreasing priority:
accuracy of the last registration (lower is better)
time elapsed since last registration (less is better)
time elapses since registration was checked the last time (less is better)
change of the orientation of the camera with respect to the reference star during registration (less change is better)
distance reference start to camera compares to the distance during registration (smaller distance is better)
distance of points acquired during registration with respect to the default value (less is better)
distance camera to patient during registration (less is better)
distance reference star to the patient head during registration (less is better)
orientation of the reference star with respect to the camera during registration (pane of the tool perpendicular to the camera is the ideal case)
relative movement of the reference star with respect to the camera during the registration (less is better)
type of data set used (e.g. CT is better than MR)
if the field of surgery is known (e.g. entered by the customer): comparison of the area with the reliability map—are they in the “reliable” area?

The n-dimensional image of a body is registered when the spatial location of each point of an actual object within a space, for example a body part in an operating theatre, is assigned an image data point of an image (CT, MR, etc.) stored in a navigation system.

Input parameters not directly related to the actual system accuracy could still be determined, recorded and compared to previous results and or default values defined by the manufacturer of the software. Examples are:
number of interactions with the software compared to a default value that is expected to be required for the surgery
duration of the system setup
total time of the surgery
changes of the position of the devices and/or components during the surgery
how often was the tool changed
which tool geometry was used for how long
number of attempts to perform a certain task, e.g. to place a certain tool

Orthopaedic surgeries are often standardized procedures and the goal of many surgeons is to minimize the duration of the surgery at a given level of precision. Examples for the weighting factor with decreasing priority could be:
accuracy of the registration relative to a predefined threshold value
precision of the alignment of the implant
time elapsed since registration was done (if a certain threshold is exceeded)
time elapsed since registration was checked the last time (less is better)
total time of the surgery
number of interactions with the software compared to a default value that is expected to be required for the surgery

The criteria not used for the assessment could still be calculated, recorded and compared to previous results and/or default values defined by the manufacturer of the software. Examples are:
accuracy of the registration (even when the predefined threshold value is exceeded)
distance camera to patient during registration (less is better)
distance reference star to the patient head during registration (less is better)
change of the orientation of the camera with respect to the reference star during registration (less change is better)
relative orientation of the reference star with respect to the camera during registration
relative movement of the reference star with respect to the camera during the registration (less is better)
time elapsed since registration was checked the last time (less is better)
changes of the position of the devices and/or components during the surgery
how often was the tool changed
which tool geometry was used for how long

The weighting factors relevant for the system accuracy could be used to compute the actual system accuracy. Besides that, the only status information the accuracy of the system could be computed as a function of time, i.e. how it evolved during the surgery. The computation could also determine the external factors that negatively influenced the system accuracy. This information could be further processed and provided to the user.

The output is preferably stored in and compared with the contents of a database. Thereby, a user may obtain feedback on his performance relative to previous surgeries which he has performed and may also receive information on how he compares to other surgeons the performed similar surgeries before. Access to this database is preferably provided using the personal identification of the surgeon. More specifically, users from different fields of surgery are preferably grouped into communities. A system to handle these data is embodied but not limited to the Quentry network provided by the applicant, Brainlab AG, essentials of which being described in the applicant's patent applications PCT/EP2011/054839 and PCT/EP2011/054833. The entire contents of both these applications is incorporated into the present disclosure by reference.

The output is preferably provided online during the surgery and/or as a summary after the surgery is completed. Moreover, the output can be customized for the different user groups comprising for example a group of surgeons and a group OR personnel.

The output for the surgeon could be but is not limited to
overall quality of the surgery computed from the various input parameters
accuracy of the registration
maximum time between the registration and the conformation of the accuracy
total duration of the surgery
number of interactions with the software
time it took to setup the system
comparison of his set up to the ideal setup and/or previous setups he used/other surgeons used
suggestion on how to improve (e.g. an animation of the surgery could be shown together with the improvement suggestions)
statistics on key numeric values like
   average accuracy from previous surgeries versus actual accuracy
   accuracy as a function of system usages
   accuracy versus his individual/community high score
   average duration from previous surgeries versus actual duration
graphical visualization of the results

Besides this, the output could also include suggestion and comments from other surgeons for specific tasks, on system setup and on what they did to further improve the overall accuracy.

Some part of the output could be created like the results of a video game showing a world-wide ranking etc. This could be combined with
extra credits for a very good registration
extra credits for customers that met the service intervals
a statistics on system usage per week/month/year and extra credits if a predefine minimum number of system usages is exceeded

It might even contain different levels like beginners, advanced and professionals.

The output preferably includes information about the OR personnel using the system. For example, a specific output is created for the person setting up the system before surgery commences in order to provide with the following information:
time it took to setup the system
quality of the first alignment of the camera versus reference star
suggestions on improvements (e.g. by suggesting to use the laser button for the rough alignment)
comparison of his set up to the ideal setup and or to previous setup he used/other customers used

This information will enable the OR staff to increase the efficiency when setting up the system.

In the following, the invention will be described with reference to the figures, which describe embodiments of the invention without limiting the invention to the contents of the specific figure and corresponding invention, wherein

FIG. 1 shows the general geometry for determining a three-dimensional uncertainty of the position of a marker sphere assuming a predetermined uncertainty of the position of one detection unit belonging to a camera which is part of a navigation system;

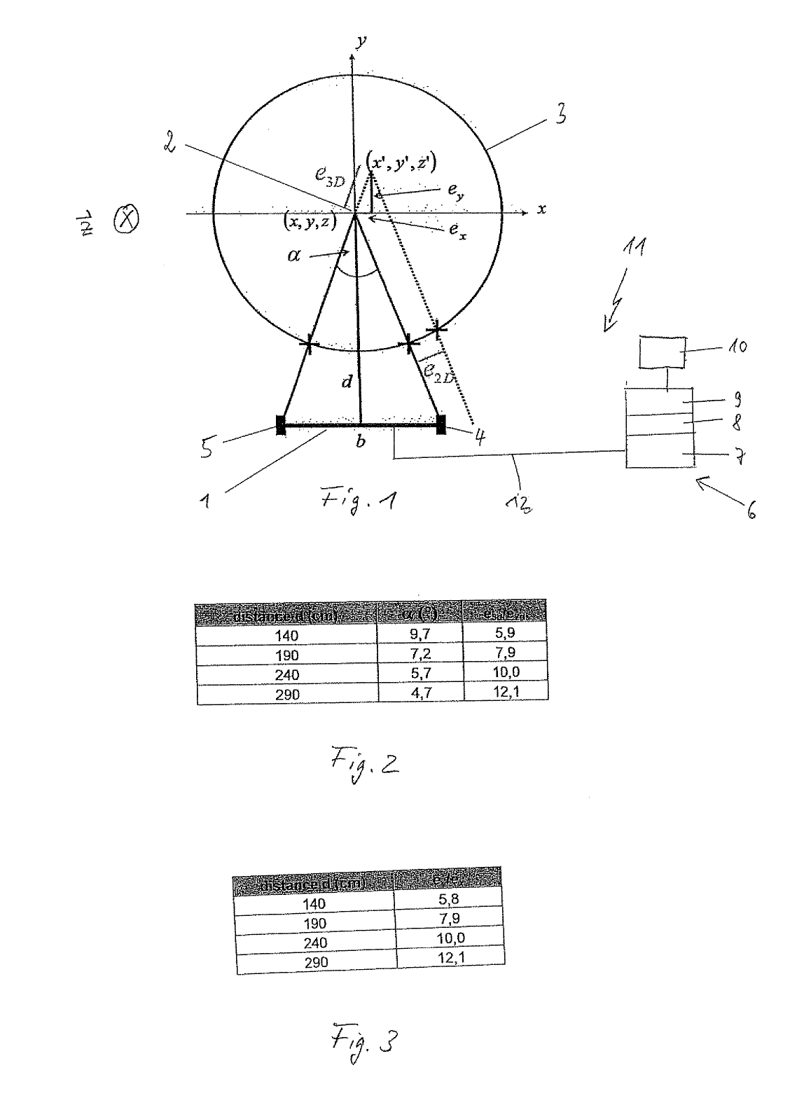

FIG. 2 is a table of numeric values for the ratio between the uncertainty of the camera position and the uncertainty of the marker position in dependency on a distance of the marker from the camera plane;

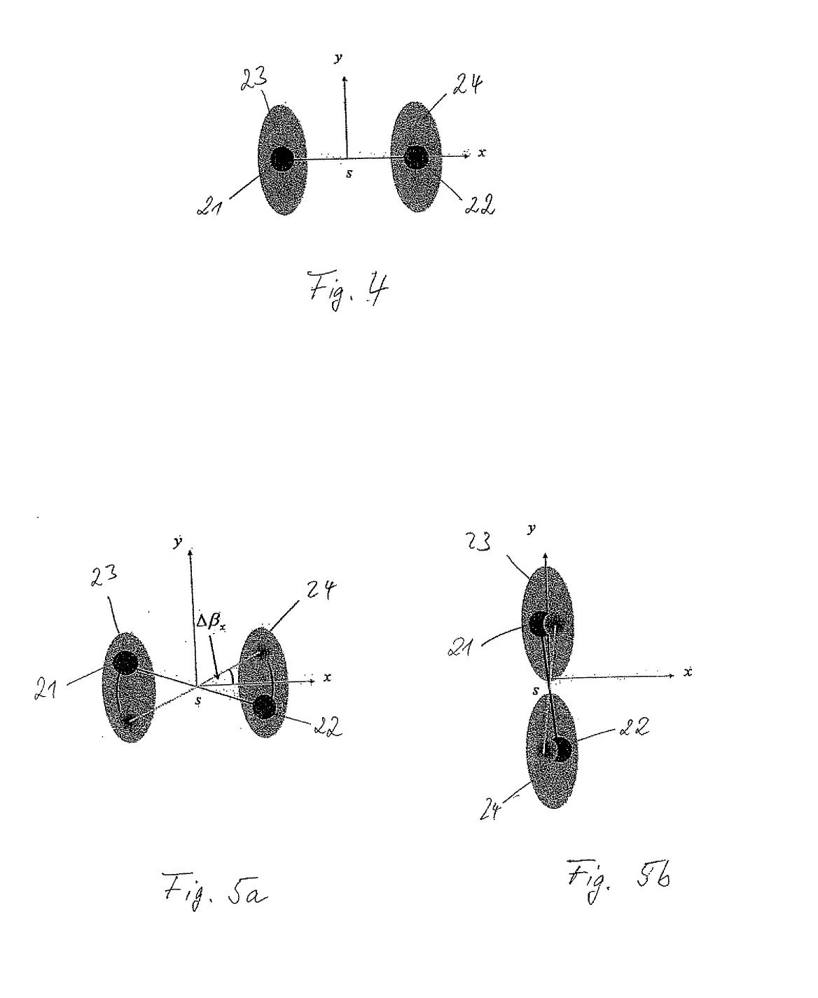

FIG. 3 is a table showing the dependency of a ratio of the errors of the marker position in different dimensions on the distance of the marker from the camera plane;

FIG. 4 is a representation of the individual uncertainties of the positions of two marker spheres;

FIGS. 5a and 5b are representations of the individual uncertainties of two marker spheres attached to an elongated tool in two dimensions and in two different orientations of the tool.

With reference to FIG. 1, the general geometry is explained which serves as a basis for the inventive method of determining the position of a medical device by using a navigation system 11. The navigation system 11 comprises a stereotactic camera 1 as a detection device having two detection units embodied by two optical sensors 4, 5. The optical sensors 4, 5 are spaced from one another by a distance b (representing the detection device geometry information in the language of claim 1) and lie in a detection plane which is parallel to the xz-plane of a Cartesian coordinate system describing the tracking volume 3 in which a marker sphere 2 (represented by a point lying in the origin of the coordinate system) is tracked by the navigation system 11. The navigation system 11 in particular comprises the camera 1, a computer 6 to which the camera 1 is connected via a data line 12 and a monitor 10 for output of visual information to an operator. The computer 6 comprises a processor (CPU) 7, a volatile memory (RAM) 8 and preferably a hard disk drive 9 for processing the positional information of the marker sphere 2 received by the camera 1 (in particular, its optical sensors 4, 5) as reflexions of electromagnetic radiation (in particular, in the infrared wavelength) from the surface of the marker sphere 2. The electromagnetic radiation is transmitted by an emission device which is not shown in FIG. 1. For example, the emission device may be part of the camera 1 and located in the detection plane.

In the setup of FIG. 1, it is assumed that the surface relative position information describes a rectangular position/arrangement of the detection surface and the uncertainty surface relative to each other.

The position of the marker sphere (i.e. the position of the medical device in the language of claim 1) in the coordinate system is defined by three-dimensional coordinates (x, y, z). The position (x, y, z) is represented by the medial device position information (in the language of claim 1) and is associated with a three-dimensional error (e_{3D}) in the xy-plane. e_{3D} (and its components eₓ and e_{y}) represents the medical device position uncertainty (in the language of claim 1) and depends on an uncertainty e_{2D} in the position of one of the optical sensors 4, 5 (in this case, of the optical sensor 4) and thereby also on the length of b. In two dimensions, e_{3D} can be divided into specific contributions to e_{3D} in the y-direction (e_{y}) and in the x-direction (e). If the position error e_{3D} of the marker sphere 2 is added to its nominal position (x, y, z), the position of the marker sphere 2 will be determined to be at (x′, y′, z′). In other words, (x′, y′, z′)=(x, y, z)+e_{3D}. e_{2D} represents the detection device geometry uncertainty information (in the language of claim 1) and is preferably predetermined and known from the operating parameters of the camera 1. The marker sphere 2 is located at the distance d from the detection plane which runs to the positions of the optical sensors 4, 5 in a direction perpendicular to the viewing direction of the optical sensors 4, 5. The numerical value of d can be determined by a foreknown value of b by the following formula:

$$tan ( \frac{α}{2} ) = \frac{b / 2}{d} \qquad ( 1 )$$

The value for a can be determined from the reflexion signal received from the marker sphere 2 by each one of the optical sensors 4, 5. Based on such knowledge, the three-dimensional error e_{3D} associated with the position of the marker sphere 2 can be determined based on the following equation:

$${e}_{3 d} = \frac{{e}_{3 d}}{sin α} . \qquad ( 2 )$$

Based on the angular relations for the x- and y-direction components of e_{3D}, one determines that

$$tan ( \frac{α}{2} ) = \frac{{e}_{x}}{{e}_{3 d}}$$

$$and$$

$$cos ( \frac{α}{2} ) = \frac{{e}_{y}}{{e}_{3 d}} .$$

The relationship between the x- and y-components of e_{3D} can therefore be stated as follows:

$$tan ( \frac{α}{2} ) = \frac{sin ( α / 2 )}{cos ( α / 2 )} = \frac{{e}_{x}}{{e}_{y}} .$$

By comparison with equation (1), the following relation is obtained:

$$⇒ \frac{b / 2}{d} = \frac{{e}_{x}}{{e}_{y}} \\ ⇒ {e}_{y} = \frac{{e}_{x} \times 2}{b} \times d . \qquad ( 3 )$$

Equation (3) shows that for a given value of the base separation b, the uncertainty in the plane perpendicular to the detection plane, i. e. in the uncertainty plane identical or parallel to the xy-plane of the coordinate system, will increase with increasing distance d. FIG. 3 shows the ratio e_{y}/eₓ for different distances at a given base separation of b=48 cm.

FIG. 2 shows that for a given two-dimensional uncertainty (or deviation of the real value of b from the true value of b) e_{2D}, which may be due to for example the fine sights of the optical sensor 4 (in particular, a CCD-sensor) or others uncertainties that stem from the camera, the three-dimensional deviation or uncertainty, respectively, e_{3D} depends inversely on the sine of the angle α. Thus, for smaller angles and equal two-dimensional uncertainty, the three-dimensional uncertainty will increase. In other words, with increasing distance d, the three-dimensional deviation will increase. FIG. 2 shows values for a typical base separation of b=48 cm.

According to the invention, not only an absolute value for e_{3D} can be determined. Rather, the invention also serves to determine the value of e_{3D} in dependence on the orientation of a medical tool to which for example two marker spheres 21, 22 as shown in FIG. 4 are attached. The two marker spheres are separated by a distance s and the axis connecting the two preferably lies on or at least parallel to the x-axis of the coordinate system of FIG. 4 (being equal in meaning to the coordinate system of FIG. 1). Due to the sine and cosine relations for eₓ and e_{y}, a two-dimensional ellyptical region can be defined around the nominal position of each marker sphere 21, 22, which represents a possible uncertainty region 23, 24 for the nominal position of each marker sphere 21, 22. Due to the angular relations for eₓ and e_{y}, it becomes clear that in the case of FIG. 4, the large uncertainty regions 23, 24 are due to the large uncertainty e_{y} in the y-direction. This case is examined in further detail in geometry of FIG. 5a, according to which an uncertainty of the tool orientation Δβₓ along the x-axis is determined as:

$$sin {Δβ}_{x} = \frac{{e}_{y}}{s / 2} ,$$

whereas the uncertainty for the tool orientation Δβ_{y} along the y-axis is determined as

$$sin {Δβ}_{y} = \frac{{e}_{x}}{s / 2} .$$

Therefore, the uncertainty of the tool orientation Δβ depends not only inversely on the base separation s between the two markers but also on the orientation of the tool itself. Determining the orientation of the tool is thus an input parameter which is well-suited to determine the accuracy of the system or of application of the system, respectively. For example, FIG. 5b shows the case in which the tool is generally oriented along the y-axis of the coordinate system of FIG. 4, whereby the uncertainty associated with the tool orientation Δβ becomes smaller than in the case of FIG. 5a, in which the tool is generally oriented along the x-axis of the coordinate system. In general, the uncertainty associated with the tool orientation Δβ is illustrated by FIGS. 5a and 5b as an angle relative to the x-axis to the axis connecting the position of the two marker spheres 21, 22 in the case of a maximum outlying actual position of the marker spheres 21, 22 on the longer semi-axes of the uncertainty regions 23, 24.

The above explanation shows that, in real setups where two-dimensional uncertainties must be expected, the distance d has a major influence on the accuracy of determining the position of the marker sphere 2. The larger d becomes, the larger e_{3D} gets. If different operators using the navigation system 11 are to be ranked, their perfomance can be sorted by achieved values for e_{3D}. From the aforementioned equations, it becomes clear that the skill operator highly depends on choosing the correct distance d and therefore on correctly placing the camera 1 relative to for example a surgical situs in which the marker sphere 2 is located.

The above also shows that the uncertainty e_{3D} associated with a position of a single marker sphere 2 is largest in the direction perpendicular to the detection plane. This leads to the fact that the uncertainty associated with the orientation of a tool Δβ depends on the orientation of the tool with respect to the camera 1. Thus, both the parameters determining the distance of the camera to a marker sphere and the orientation of the camera relative to the marker sphere are important parameters for determining the accuracy of navigation in a navigated medical procedure. The operator ranking may for example be constituted such that, the lower the achieved value for e_{3D} is, the better the associated operator's ranking is. In particular, small values for e_{3D} may be used to describe a high accuracy of navigation, whereas larger values for e_{3D} may be used for describing a less accurate navigation.

1. A data processing method of determining the orientation, of a medical device relative to a stereotactic detection device for detecting the position of the medical device, the data processing method being executable by a computer and comprising the following steps:
a) acquiring medical device position data comprising medical device position information describing the locations of at least two detection features of the medical device relative to a detection surface in which the detection device is located;
b) acquiring detection device geometry data comprising detection device geometry information describing a geometry of the detection device;
c) acquiring detection device geometry uncertainty data comprising detection device geometry uncertainty information describing a detection device geometry uncertainty associated with the geometry of the detection device described by the detection device geometry information, wherein the detection device geometry uncertainty is defined relative to an uncertainty surface;
d) acquiring surface relative position data comprising surface relative position information describing the relative position between the detection surface and the uncertainty surface;
e) determining, based on the medical device position data and the detection device geometry data and the detection device geometry uncertainty data and the surface relative position data, medical device position uncertainty data comprising medical device position uncertainty information describing a medical device position uncertainty associated with the orientation of the medical device relative to the detection device, wherein the medical device position uncertainty is defined relative to the uncertainty surface, wherein determining the medical device position uncertainty data comprises determining the uncertainty of the locations of the at least two detection features of the medical device.

2. (canceled)

3. (canceled)

4. (canceled)

5. The method according to the claim 1, wherein the uncertainty of the orientation is determined relative to at least one of a direction parallel to the detection plane and a direction perpendicular to the detection plane, in particular based on determining at least one angle between a straight line connecting the detection features and at least one of these directions.

6. The method according to claim 5, wherein the position of the medical device relative to the detection device is changed based on the medical device position uncertainty data, in particular in order to minimize the medical device position uncertainty.

7. The method according to claim 1, wherein a graphical representation of the medical device position uncertainty information is output.

8. The method according to claim 1, wherein the detection device is a camera and wherein the medical device comprises or is at least one marker.

9. The method according to claim 1, wherein the medical device position uncertainty data is determined based on acquiring the medical device position data for at least two positions of the medical device relative to the detection device, and wherein the medical device position uncertainty data determined for one of those positions is compared to the medical device position uncertainty data determined for another one of those positions.

10. The method according to claim 9, wherein the medical device position uncertainty information determined for each of the at least two positions is sorted in an incremental, in particular an increasing or decreasing, order.

11. The method according to claim 10, wherein an optimal position of the medical device and relative to detection device is selected based on the medical device position uncertainty data determined for the at least two arrangements, wherein the optimal position in particular is a position for which a minimum of the medical device position uncertainty has been determined.

12. The method according to claim 1, wherein weights are applied to the medical device position uncertainty depending on the position of the information in the order,
wherein the medical device position uncertainty information determined for each of the at least two positions is sorted in an incremental, in particular an increasing or decreasing, order.

13. A program which, when running on a computer or when loaded onto a computer, causes the computer to perform the following steps:
a) acquiring medical device position data comprising medical device position information describing the locations of at least two detection features of the medical device relative to a detection surface in which the detection device is located;
b) acquiring detection device geometry data comprising detection device geometry information describing a geometry of the detection device;
c) acquiring detection device geometry uncertainty data comprising detection device geometry uncertainty information describing a detection device geometry uncertainty associated with the geometry of the detection device described by the detection device geometry information, wherein the detection device geometry uncertainty is defined relative to an uncertainty surface;
d) acquiring surface relative position data comprising surface relative position information describing the relative position between the detection surface and the uncertainty surface;
e) determining, based on the medical device position data and the detection device geometry data and the detection device geometry uncertainty data and the surface relative position data, medical device position uncertainty data comprising medical device position uncertainty information describing a medical device position uncertainty associated with the orientation of the medical device relative to the detection device, wherein the medical device position uncertainty is defined relative to the uncertainty surface, wherein determining the medical device position uncertainty data comprises determining the uncertainty of the locations of the at least two detection features of the medical device.

14. A navigation system for computer-assisted surgery, comprising:
the computer of claim 13, for processing the medical device position data, the detection device geometry data, the detection device geometry uncertainty, and the medical device position uncertainty data;
a detection device for detecting the position of the medical device;
a data interface for receiving the medical device position data from the detection device and for supplying that data to the computer; and
a user interface for receiving data from the computer in order to provide information to a user, wherein the received data are generated by the computer on the basis of the results of the processing performed by the computer.

15. A program storage medium on which the program according to claim 13 is stored in a non-transitory form.

16. A computer, in particular a cloud computer, on which the program according to claim 13 is running or into the memory of which the program according to claim 13 is loaded.

17. A digital signal wave carrying information which represents the program according to claim 13.
